(19) United States
(12) Patent Application Publication
(10) Pub. No.: US 2026/0041433 A1
(43) Pub. Date: Feb. 12, 2026

(54) Self-Adjusting Vascular Occlusion Catheter Balloon

(71) Applicant: Prytime Medical Devices, Inc., Boerne, TX (US)

(72) Inventor: Curtis J. FRANKLIN, Boerne, TX (US)

(21) Appl. No.: 19/099,811

(22) PCT Filed: Jul. 27, 2023
(86) PCT No.: PCT/US2023/028828

§ 371 Date: Jan. 30, 2025

Related U.S. Application Data
(60) Provisional application No. 63/394,336, filed on Aug. 2, 2022.

(51) Int. Cl. A61B 17/12; A61B 17/00; A61M 25/10

(52) U.S. Cl. CPC A61B 17/12136; A61B 17/12036; A61B 17/1204; A61B 17/12109; A61M 25/1002; A61B 2017/00557; A61B 2017/1205; A61M 2025/1004; A61M 2025/1052; A61M 2205/3344; A61M 2210/12

(57) ABSTRACT

A vascular occlusion catheter is configured for insertion, and at least partial inflation, into a targel blood vessel. The vascular occlusion catheter includes a proximal catheter shaft, a distal catheter shaft, and a semi-compliant or non-compliant occlusion balloon mounted at a proximal side thereof to the proximal catheter shaft and mounted at a distal side thereof to the distal catheter shaft. A central catheter shaft extends through the proximal catheter shaft, the occlusion balloon and into the distal catheter shaft. The central catheter shaft is constructed as the primary load-bearing chassis of the vascular occlusion catheter. The occlusion balloon defines a blown diameter between approximately twenty-five and approximately thirty-five millimeters and the occlusion balloon defines a double-wall thickness of between approximately 0.0003″ and approximately 0.0020″.

### CROSS-REFERENCE TO RELATED APPLICATION

This application is a Section 371 of International Application No. PCT/US2023/028828, filed Jul. 27, 2023, which was published in the English language on Feb. 8, 2024, under International Publication No. WO 2024/030319 A1, which claims priority from similarly-titled U.S. Provisional Ser. No. 63/394,336 , filed Aug. 2, 2022, the entire contents of which are incorporated by reference herein.

### BACKGROUND OF THE DISCLOSURE

The present disclosure generally relates to vascular occlusion catheters, and, more particularly, to vascular occlusion catheter balloons.

Endovascular occlusion, such as resuscitative endovascular balloon occlusion of the aorta (“REBOA”) or partial endovascular balloon occlusion of the aorta (“P-REBOA”), is a procedure in which a blood vessel is fully or at least partially occluded in order to restrict blood flow downstream of the occlusion site. Partial occlusion or P-REBOA is advantageous at least to limit blood flow to organs and tissue downstream of the occlusion site while mitigating the risk of ischemia. That is, partial occlusion may be desirable to provide at least partial blood flow to portions of the patient's body downstream of the occlusion balloon. Partial perfusion past the occlusion balloon can provide the benefits of focusing or directing a majority of blood flow to the brain, heart and lungs or other upstream portions of the patient, but also potentially increasing the amount of time the occlusion balloon can be implanted in the patient, by providing at least partial blood flow to the patient's organs downstream of the occlusion balloon, such as to the patient's liver, digestive tract, kidneys and legs.

In addition to monitoring the duration of occlusion, the degree of occlusion and the location of the balloon must also be monitored. Loss of contact with the inner surface of a vessel by the occlusion balloon, for example, may also result in decreased occlusion effectiveness and/or the occlusion balloon and attached catheter may be moved downstream in the vessel, thereby moving the occlusion balloon out of its preferred placement. For example, it is known that a sudden change in aortic pressure is met by a rapid adjustment of vascular resistance (via vasoconstriction or vasodilation) in the body's natural effort to maintain appropriate blood flow, i.e., autoregulation. In the case of trauma/hemorrhage, for example, the aorta is likely to contract in an effort to maintain blood pressure. Upon insertion of an occlusion balloon catheter into the proper location along the aorta to minimize the hemorrhage and focus the majority of blood flow to the upstream organs, the aorta may, in turn, further autoregulate by beginning to relax the vasoconstriction, thereby changing its inner diameter. Studies have shown that autoregulation may result in aortic internal diameter change up to approximately thirty percent (30%). Such aortic changes may impact the apposition between the balloon and the vascular wall, and, in turn, negatively affect the degree of occlusion, thereby requiring continued or periodic intervention by the user or machine, e.g., to adjust balloon volume and/or location, to maintain the desired degree of occlusion.

It would, therefore, be desirable to design, develop and implement an occlusion balloon catheter that accommodates dynamic vasculature changes, e.g., natural vasculature autoregulation via vasoconstriction or vasodilation, and maintains contact with the vessel at substantially the desired position, thereby reducing or eliminating the need for continued or periodic intervention by the user or automatic titration of the balloon via a machine and controls.

### BRIEF SUMMARY OF THE DISCLOSURE

Briefly stated, one aspect of the present disclosure is directed to a vascular occlusion catheter configured for insertion, and at least partial inflation, into a targel blood vessel. The vascular occlusion catheter includes a proximal catheter shaft, a distal catheter shaft, and a semi-compliant or non-compliant occlusion balloon mounted at a proximal side thereof to the proximal catheter shaft and mounted at a distal side thereof to the distal catheter shaft. A central catheter shaft extends through the proximal catheter shaft, the occlusion balloon and into the distal catheter shaft. The central catheter shaft is constructed as the primary load-bearing chassis of the vascular occlusion catheter. The occlusion balloon defines a blown diameter between approximately twenty-five and approximately thirty-five millimeters and the occlusion balloon defines a double-wall thickness of between approximately 0.0003″ and approximately 0.0020″.

In one configuration, the occlusion balloon defines a functional working length of approximately 80 mm or less.

In any of the previous configurations, the proximal catheter shaft, the occlusion balloon, and the distal catheter shaft have a greatest outer diameter of seven French (7 Fr) or less in an uninflated condition.

In any of the previous configurations, upon inflation of the occlusion balloon into apposition with the target blood vessel, the occlusion balloon is configured to automatically adjust shape in response to diametric autoregulation of the target blood vessel, and, thereby, continue in apposition therewith.

In any of the previous configurations, upon inflation of the occlusion balloon into apposition with the target blood vessel, the occlusion balloon is configured to undergo intra-balloon inflation medium volume redistributrion, without altering intra-balloon inflation medium volume, in response to diametric autoregulation of the target blood vessel, and, thereby, automatically continue in apposition therewith.

In any of the previous configurations, upon inflation of the occlusion balloon into apposition with the target blood vessel, the occlusion balloon is configured to automatically transition from defining a generally cylindrically-shaped mid-portion bookended by generally conically-shaped portions to a tear-drop shape at a downsream end of the occlusion balloon in response diametric autoregulation of the target blood vessel into a normotensive state.

In any of the previous configurations, the occlusion balloon is configured such that an outer surface of the occlusion balloon comes into full diametric apposition with an inner surface of the target blood vessel upon partial inflation of the occlusion balloon, whereby folds are formed in the outer surface of the occlusion balloon, the folds defining flow channels with inner surfaces of the target blood vessel or with portions of the outer surface of the occlusion balloon that allow partial blood flow past the occlusion balloon.

Briefly stated, one aspect of the present disclosure is directed to a method of using the vascular occlusion catheter of any one of the previous configurations. The method includes the steps of inserting the vascular occlusion catheter in an uninflated condition into a desired location of the target blood vessel; and inflating the occlusion balloon, with an appropriate volume of inflation medium, into a partially inflated state, wherein an outer surface of the occlusion balloon comes into substantially full diametric apposition with an inside wall of the target vessel and a state of partial occlusion of the target blood vessel is established, whereby the occlusion balloon is configured to autonomously, substantially maintain the state of partial occlusion in response to autoregulation of the target blood vessel.

In one method configuration, the inserting step includes advancing the distal catheter shaft, the occlusion balloon and the proximal catheter shaft through an introducer sheath having an inner introducer diameter of 7 Fr or less.

In any of the previous method configurations, the inflating step includes inflating the occlusion balloon to an intra-balloon pressure of between approximately 60 mmHg and approximately 150 mmHg.

In any of the previous method configurations, the inflating step includes folds being formed in the outer surface of the occlusion balloon, the folds defining flow channels with inner surfaces of the target blood vessel or with portions of the outer surface of the occlusion balloon that allow partial blood flow past the occlusion balloon.

In any of the previous method configurations, the occlusion balloon is configured to autonomously, substantially maintain the state of partial occlusion in response to autoregulation of the target blood vessel via automatically adjusting shape in response to diametric autoregulation of the target blood vessel.

In any of the previous method configurations, the occlusion balloon is configured to autonomously, substantially maintain the state of partial occlusion in response to autoregulation of the target blood vessel via intra-balloon inflation medium volume redistributrion, without altering intra-balloon inflation medium volume.

In any of the previous method configurations, the occlusion balloon is configured to autonomously, substantially maintain the state of partial occlusion in response to autoregulation of the target blood vessel via automatically transitioning from defining a generally cylindrically-shaped mid-portion bookended by generally conically-shaped portions to a tear-drop shape at a downsream end of the occlusion balloon.

### BRIEF DESCRIPTION OF THE SEVERAL VIEWS OF THE DRAWINGS

The following description of the disclosure will be better understood when read in conjunction with the appended drawings. It should be understood, however, that the disclosure is not limited to the precise arrangements and instrumentalities shown. In the drawings:

FIG. 1 is a perspective view of a vascular occlusion catheter according to an embodiment of the present disclosure;

FIG. 2 is a partial, cross-sectional view of the vascular occlusion catheter of FIG. 1, in a simulated target vessel, taken along the sectional line 2-2 of FIG. 1;

FIG. 3 is a cross-sectional view of the occlusion balloon of FIG. 1 inflated to a partially occluded configuration, taken along line 3-3 of FIG. 2;

FIG. 4 is a cross-sectional view of the occlusion balloon of FIG. 1 inflated to a full occlusion configuration, taken along line 3-3 of FIG. 2;

FIG. 5 is an elevational view of the occlusion balloon of FIG. 1 in an inflated configuration;

FIG. 6A is a partial plan view of the vascular occlusion catheter of FIG. 1 in apposition with a simulated target vessel having a constricted diameter;

FIG. 6B is a partial plan view of the vascular occlusion catheter of FIG. 6A, having a self-adjusted shape to remain in apposition with the simulated target vessel having a relaxed diameter relative to FIG. 6A; and

FIG. 6C is a partial plan view of the vascular occlusion catheter of FIG. 6A, having a further self-adjusted shape to remain in apposition with the simulated target vessel having a further relaxed diameter relative to FIGS. 6A and 6B.

### DETAILED DESCRIPTION OF THE DISCLOSURE

Certain terminology is used in the following description for convenience only and is not limiting. The words “lower,” “bottom,” “upper” and “top” designate directions in the drawings to which reference is made. The words “inwardly,” “outwardly,” “upwardly” and “downwardly” refer to directions toward and away from, respectively, the geometric center of the vascular occlusion catheter, and designated parts thereof, in accordance with the present disclosure. In describing the vascular occlusion catheter, the term proximal is used in relation to the end of the catheter closer to the user and the term distal is used in relation to the end of the catheter further from the user. The terms upstream and downstream are used in relation to the direction of blood flow within a vessel. Unless specifically set forth herein, the terms “a,” “an” and “the” are not limited to one element, but instead should be read as meaning “at least one.” The terminology includes the words noted above, derivatives thereof and words of similar import.

It should also be understood that the terms “about,” “approximately,” “generally,” “substantially” and like terms, used herein when referring to a dimension or characteristic of a component of the disclosure, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

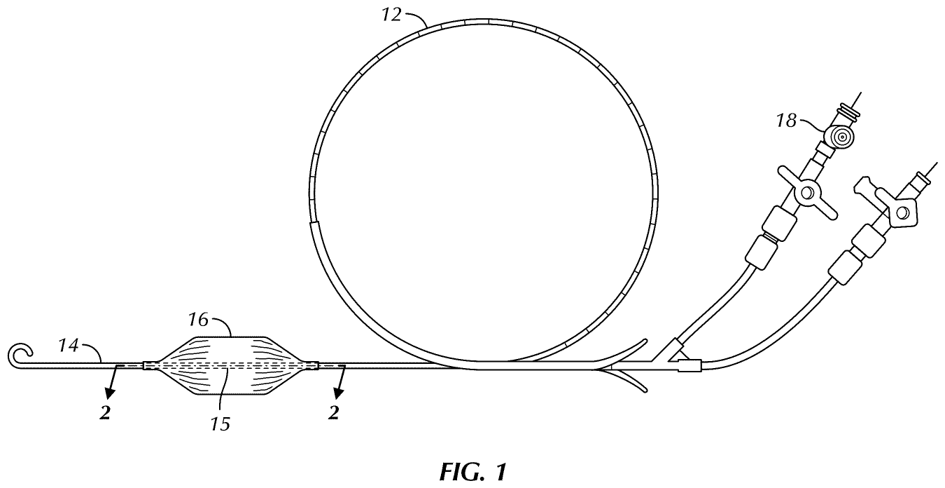

Referring to the drawings in detail, wherein like numerals indicate like elements throughout, there is shown in FIGS. 1-6C a vascular occlusion catheter, generally designated 10, in accordance with an embodiment of the present disclosure. Generally, the vascular occlusion catheter 10 includes at least a proximal catheter shaft 12, a distal catheter shaft 14 and an occlusion balloon 16 mounted to the proximal and distal catheter shafts 12, 14. Other exemplary vascular occlusion catheters, having further optional components, are described in International Patent Application Publication No. WO 2020/033372, titled “System and Method for Low Profile Occlusion Balloon Catheter” and in International Patent Application Publication No. WO 2022/197895, titled “Vascular Occlusion Catheter”, the entire contents of each of which are expressly incorporated by reference herein.

A central catheter shaft 15 may extend through the proximal catheter shaft 12, the occlusion balloon 16 and into the distal catheter shaft 14. In some configurations, the central catheter shaft 15 may form the structural backbone/chassis of the catheter 10. Stated differently, the central catheter shaft 15 may form the primary load-bearing chassis/framework of the catheter 10, eliminating the requirement for a separate guidewire to provide such load bearing stability (though a guidewire may optionally be utilized if desired).

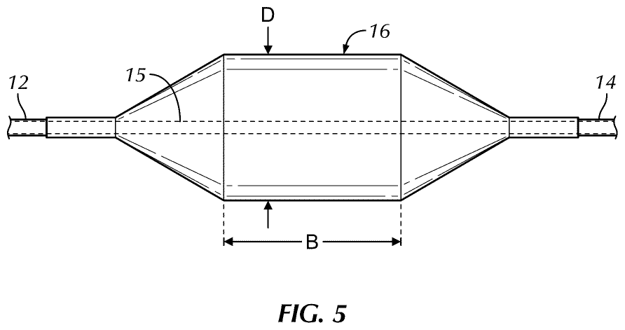

In one configuration, the occlusion balloon 16 may be comprised of a large/oversized outer, blown diameter D (see FIG. 5), semi-compliant or substantially non-compliant balloon 16. In the context of the present disclosure, a non-compliant or substantially non-compliant balloon generally has growth of approximately two to seven percent (2-7%) within the working range (balloon pressure) when inflated, a semi-compliant balloon has growth of approximately seven to twenty percent (7-20%) within the working range (balloon pressure) when inflated and a compliant balloon has growth of approximately greater than twenty percent (20%+) within the working range (balloon pressure) when inflated. Compliant balloons may have growth of approximately one hundred to three hundred percent (100-300%) within the working range (balloon pressure) when inflated.

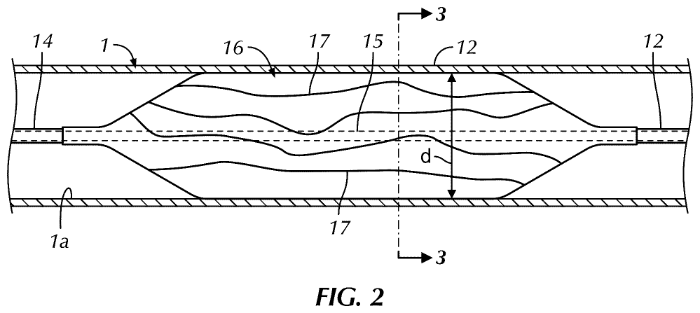

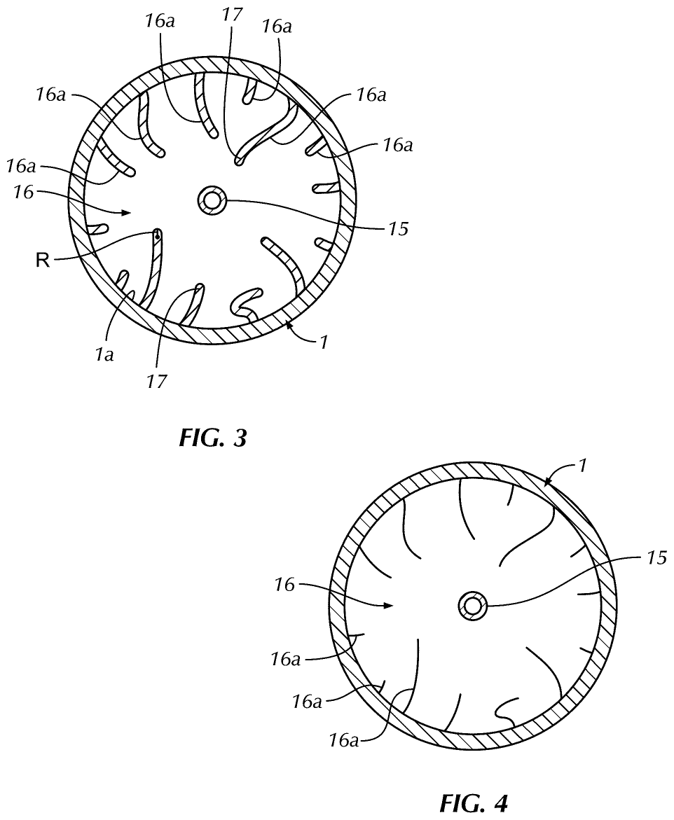

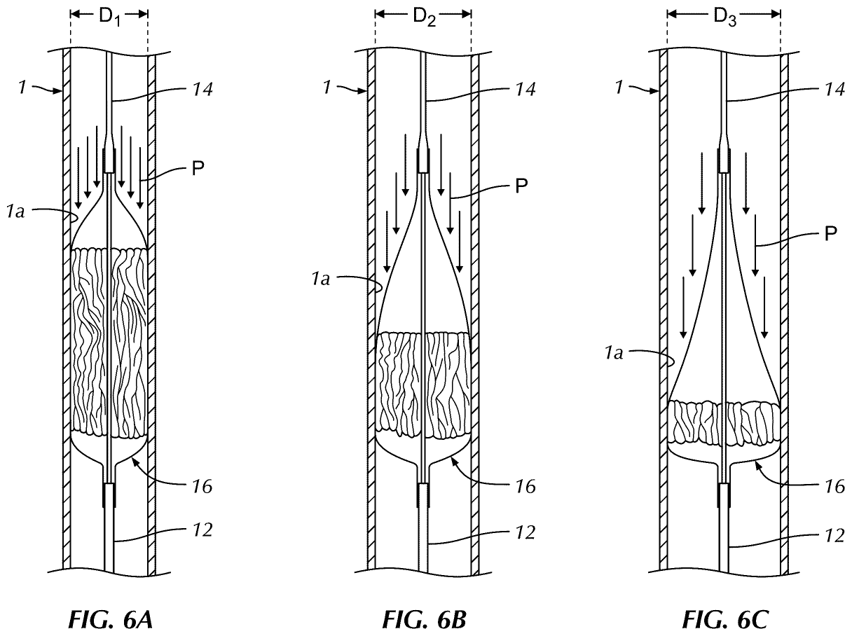

The occlusion balloon 16 may be “oversized”, i.e., define a blown diameter D that is larger than a diameter D1, D2 or D3 of the target, i.e., destination, blood vessel 1 (as described further below with respect to FIGS. 6A-6C) into which the balloon 16 is inserted and inflated for occlusion. In one configuration, the occlusion balloon 16 may define a blown diameter D between approximately twenty-five and approximately thirty-five millimeters (˜25-35mm), which is configured to be between approximately ten percent and approximately sixty percent (10-60%) larger than the diameter D3 of the target vessel 1, e.g., the aorta, in a normotensive state. The balloon 16 is, therefore, only partially inflated when its outer surface comes into substantially full diametric contact/apposition with the inside wall 1a of the target vessel 1 while folds 16a (see FIG. 3) remain at the outer surface of the balloon 16. In this partially inflated configuration, the occlusion balloon 16 has a partially inflated diameter d (see FIG. 2), wherein folds 16a are formed in the peripheral surface of the occlusion balloon 16. The open/un-collapsed folds 16a, in combination with the opposing inner wall la of the vessel 1 or with radially overlying portions of the outer surface of the balloon 16, result in flow channels 17 extending along the length of the occlussion balloon 16 (see FIG. 2) that allow partial perfusion or blood flow past the balloon 16 as a result of the blood pressure within the vessel 1. The cross-hatching within the folds 16a of FIG. 3 represents blood or fluid flowing through the flow channels 17, although the folds 16a may otherwise be open in this partially inflated configuration. As shown in FIG. 4, the balloon 16 (already in substantially full diametric apposition with the inside wall 1a of the target vessel 1) may be further inflated (relative to the partially inflated configuration), such that the sides of the folds 16a collapse upon each other and seal off the flow channels 17, thereby providing full occlusion of the vessel 1.

Balloon wall thickness of a semi-compliant or substantially non-compliant balloon 16 is generally thinner than a comparable compliant balloon because the “less stretchy” nature of the semi-compliant or substantially non-compliant balloon material allows the balloon 16 to be thinner and still withstand the force of blood trying to push the balloon 16 downstream. Conversely, a compliant balloon generally requires a thicker wall as a thinner wall may sag under the force/pressure of blood thereon. In a semi-compliant or substantially non-compliant configuration of the occlusion balloon 16, for example, the balloon 16 may define a double-wall thickness (“DWT”) of between approximately 0.0003″ and approximately 0.0020″, such as, for example, without limitation, approximately, 0.0009″. Conversely, in a compliant configuration of the occlusion balloon 16, the balloon may define a DWT of between approximately 0.0015″ and approximately 0.0050″, such as, for example, without limitation, approximately 0.0025″. Thus, balloon wall thickness of a semi-compliant or substantially non-compliant balloon 16 may be approximately three times thinner than that of a compliant balloon 16. As should be understood by those of ordinary skill in the art, directly and accurately measuring single wall thickness of a thin-walled balloon is very challenging, and, therefore, DWT is a measurement obtained by compressing the two opposing balloon walls together, i.e., placing the two opposing balloon walls in facing contact with one another, and measuring the combined thickness of both opposing walls of the body of the balloon.

Employing such a thin-walled, semi-compliant or substantially non-compliant occlusion balloon 16 facilitates the vascular occlusion catheter 10 to exhibit a “low-profile”, e.g., having a cross-sectional profile of seven French (7 Fr) or less in an uninflated condition, such as, six French (6 Fr), five French (5 Fr) or four French (4 Fr). Accordingly, for example, the distal catheter shaft 14, the occlusion balloon 16 in the folded configuration and the proximal catheter shaft 12 are movable through an introducer sheath (not shown) having an inner introducer diameter of 7 Fr or less for introduction into the target blood vessel. Employing such a thin-walled, semi-compliant or substantially non-compliant occlusion balloon 16 also results in readily enabling the oversized balloon 16 to change shape and fold over itself and form the folds 16a with a tight radius of curvature R at the overlapping inner tip (see FIG. 3). Such a tight radius of curvature R is particularly advantageous to enable the portions of the balloon wall forming folds 16a to readily seal/collapse against one another, particularly at the radially interior periphery of the folds 16a, to close the flow channels 17 and enable substantially full occlusion, without requiring a significant increase in intra-balloon pressure and/or volume (from the partially inflated configuration) to force the folds 16a to seal.

Generally, an occlusion balloon defines a molded working length B (see FIG. 5), i.e., the length of the generally cylindrical body portion of the balloon intended to contact the target vessel wall 1a. The occlusion balloon 16, being oversized relative to the target vessel 1, however, also contacts the target vessel wall 1a with portions of the generally conically shaped end portions thereof (bookending the central cylindrical body), thereby defining a longer functional working length, i.e., the length of the target vessel 1 that would be contacted and occluded by any portion of the oversized balloon 16, of approximately 80 mm or less, such as, for example, without limitation, approximately 60 mm or less. Advantageously, the longer the functional working length of the occlusion balloon 16, and in turn, the increased contact area with the target vessel wall 1a, the less sensitive the degree of occlusion of the balloon 16 to intra-balloon volume changes, thereby requiring less precision by the user when introducing inflation medium into the balloon 16 without sacrificing partial and or full occlusion effectiveness. Stated differently, the longer the functional working length of the occlusion balloon 16, the greater the range of acceptable intra-balloon pressure and volume that results in partial occlusion as comparatively greater lengths of the balloon 16 makes changes to the degree of occlusion more gradual. As should be understood by those of ordinary skill in the art, however, the functional working length of the occlusion balloon 16 is limited by the length of the target vessel 1, and, more importantly, by the intended target placement of the occlussion balloon 16 and length of the portion of the target vessel 1 that is acceptable to be safely occluded.

A pressure-relief or pop-off valve 18 may be positioned along the proximal catheter shaft 12 and fluidly connected with the occlusion balloon 16. In one non-limiting example, the pressure-relief valve 18 may take the form of a combination stopcock and pressure relief valve, such as described in International Patent Application Publication No. WO 2022/016109, titled “Inflation Hub for a Fluid Inflatable Apparatus”, the entire contents of which are expressly incorporated by reference herein. The pressure-relief valve 18 may be employed to prevent the occlusion balloon 16 from overinflating. That is, without the presence of a pressure-relief valve 18, an oversized, substantially non-compliant or semi-compliant balloon 16 has the ability to forcibly dilate a vessel 1 when pressure exceeding a threshold is applied, which could lead to vessel 1 rupture. The combination of the pressure-relief valve 18 and the substantially non-compliant or semi-compliant oversized occlusion balloon 16, however, is configured to enable the user to inflate the occlusion balloon 16 safely until the pressure-relief valve 18 releases the inflation medium.

Advantageously, the substantially non-compliant or semi-compliant occlusion balloon 16 exhibits a slow and gradual increase in internal balloon pressure during initial balloon volume increase (inflation) and, after reaching full vessel occlusion exhibits a sharp increase in pressure with limited additional increase in balloon volume. The range of inflation fluid introduction and removal, therefore, is relatively forgiving for the substantially non-compliant or semi-compliant balloon 16 just below full occlusion pressures and volumes, when the balloon 16 is oversized for the associated vessel 1. Thus, a user is able to readily identify full occlusion. That is, the substantially non-compliant or semi-compliant occlusion balloon 16 provides a clear tactile indication to the user that the balloon 16 has come into full facing apposition with the inside surfaces 1a of the vessel 1 based on the steep pressure increase with relatively little additional inflation medium introduction into the balloon 16. This facilitates the pressure-relief valve 18 releasing pressure well below an unsafe region of inflation is reached, where rupture of the vessel 1 or the balloon 16 could potentially occur. With the occlusion balloon 16, the user is also readily able to control partial occlusion below the full occlusion range (due to the balloon's forgiving/gradual pressure curve at partial occlusion). Conversely, a compliant balloon 16 has a more consistent balloon pressure vs. balloon volume slope both below and above full occlusion when oversized for the associated vessel 1. Thus, the substantially non-compliant or semi-compliant occlusion balloon 16 provides both partial and full occlusion effectiveness and is prevented from rupture of the balloon 16 and rupture of the vessel 1 by pressure release from the pop-off or pressure-relief valve 18.

A further advantage of the substantially non-compliant or semi-compliant, oversized occlusion balloon 16 is the ability to maintain a desired setpoint/range of partial occlusion via auto-correction, i.e., without requiring the user or machine to continuosly or periodically titrate balloon volume, despite the body autonomously changing aorta diameter. That is, generally, after insertion of the vascular occlusion catheter 10 into the appropriate location within a target vessel 1, the occlusion balloon 16 is inflated to an appropriate intra-balloon volume that establishes a desired state of partial occlusion of the vessel 1 (i.e., as identified via above and/or below balloon blood pressure) (see FIG. 6A). In one configuration, the at least partially inflated, oversized occlusion balloon 16 may have a generally cylindrical mid-portion, bookended by opposing generally conical portions. Naturally, the upstream flow of blood applies a pressure P to the leading surface of the balloon 16. Due to the presence of inflation medium inside the balloon 16, the blood pressure tries to push the inflation medium downstream, which would alter the shape of the partially inflated balloon 16 toward an increasingly downstream-heavy tear-drop shape. The bulbous portion of the tear-drop contacts the blood vessel wall 1a and the oversized diameter of the balloon 16 results in the flow channels 17 (as previously described) that allow a controlled amount of flow to pass by the balloon 16.

Upon application of the vascular occlusion catheter 10 (as previously described), the patient's vessel 1 diameter D1, e.g., the aortic internal diameter, may autoregulate via vasodilation in response to the focus of blood flow to vital organs. Advantageously, the oversized balloon 16 automatically alters and/or adjusts its shape under the bias of the blood pressure P to accommodate the new diameters D2, D3 of the vessel 1, without requiring user or machine intervention to adjust balloon volume (see FIGS. 6B, 6C). Stated differently, the balloon 16 does not experience intra-balloon inflation medium volume change, but rather an intra-balloon inflation medium volume redistribution. Therefore, and as previously described, the wall thinness of the semi-compliant or substantially non-compliant, oversized balloon 16, e.g., between approximately 0.0003″ and approximately 0.0020″ as well as the relatively inelastic nature of the balloon material, in combination with the relatively low intra-balloon pressure, play a prominent role in enabling the balloon 16 to readily adjust shape. As should be understood, the relatively low intra-balloon pressure allows the balloon inflation medium to shift to the downstream end of the balloon 16. During partial occlusion, intra-balloon pressure is similar to systolic blood pressure, e.g., between approximately, 60 mmHg and approximately 150 mmHg.

As shown in FIG. 6B, vasodilation from a narrower D1 to a greater D2, results in the oversized balloon 16 correcting to a more “bottom-heavy” tear drop shape, i.e., at the downstream, proximal end of the balloon 16. That is, the volume of inflation medium within the balloon 16 is rebalanced such that an increased distal portion of the balloon 16 is narrowed relative to FIG. 6A, and may lose contact with the inner wall 1a of the vessel 1, in favor of a proximal portion of the balloon 16 being expanded with increased inflation medium relative to FIG. 6A, resulting in continued apposition with the opposing portion of the inner wall 1a of the vessel 1. Subsequently, as the vessel 1 further approaches a normotensive state, as shown in FIG. 6C, expansion of vessel diameter (vasodilation) from D2 to D3 results in the oversized ballon 16 automatically further rebalancing the volume of the inflation medium therein to a yet further “bottom-heavy”/“downstream-heavy” tear drop shape. The blood flow passing by the balloon 16 and resulting above/below balloon blood pressures remain relatively constant throughout this process depending on the dynamic nature of the patient's body.

As should be understood by those of ordinary skill in the art, vasculature may alternatively autoregulate, e.g., initially, via further vasoconstriction. Advantageously, the construction of the oversized balloon 16 also permits the balloon 16 to automatically alter and/or adjust its shape to accommodate a more constricted diameter of the vessel 1. That is, the construction of the balloon 16 permits the volume of the inflation medium within the balloon 16 to rebalance such that the balloon 16 takes on an altered shape, e.g., a longer functional working length, while maintaining the presence of the flow channels 17 extending along the length of the occlussion balloon 16, thereby maintaining blood flow past the balloon 16 from an upstream side to a downstream side, maintaining partial occlusion.

Thus, the partial occlusion stability provided by the occlusion balloon 16 construction frees up the user to perform other critical tasks required by the patient's condition, instead of constantly monitorning the balloon location within the target vessel, balloon and blood pressures, blood flow rates or other physiological characteristics of the patient to maintain a generally constant partial flow through the aorta and past the inflated balloon 16. A periodic adjustment may be selectively performed to re-optimize the partial occlusion setting during active resuscitation (i.e., blood transfusion, pharmacologic agents) but frequent titration is not required as with traditional occlusion balloons.

A further advantageous effect of the semi-compliant or substantially non-compliant material of the occlusion balloon 16 is that the occlusion balloon 16 does not receive, temporarily store and then release/impart stored above-balloon blood pressure, i.e., upstream, pulsatile blood pressure, onto the downstream blood. That is, a fully inflated compliant balloon that is fully occlusive to the target vessel 1 may nevertheless receive, temporarily store and then impart some of the upstream pressure wave onto the downstream blood pressure. This is significant because the user relies upon downstream blood pressure to assess whether blood is flowing beyond the occlusion balloon 16. Even a small amount of downstream pulsatility could be interpreted by the user that there is blood flowing beyond the balloon 16. Consequently, the user may dangerously mistake that blood is still flowing beyond the balloon, i.e., think that only partial occlusion has been achieved when in fact full occlusion has been achieved. Conversely, the semi-compliant or substantially non-compliant occlusion balloon 16 does not receive, temporarily store and then release/impart stored above-balloon blood pressure onto the downstream blood. The lack of pulsatile blood pressure below the occlusion balloon 16 when the target vessel 1 is fully occluded properly informs the user that there is no blood flow passing the balloon 16. Similarly, when pulsatile blood pressure is observed below the occlusion balloon 16, the user can be confident that some blood is flowing past the balloon 16.

It will, therefore, be appreciated by those skilled in the art that various modifications and alterations could be made to the disclosure above without departing from the broad inventive concepts thereof. Some of these have been discussed above and others will be apparent to those skilled in the art. It is understood, therefore, that this invention is not limited to the particular embodiments disclosed, but it is intended to cover modifications within the spirit and scope of the present disclosure, as set forth in the appended claims.

1. A vascular occlusion catheter configured for insertion, and at least partial inflation, into a targel blood vessel, the vascular occlusion catheter comprising:
a proximal catheter shaft;
a distal catheter shaft;
a semi-compliant or non-compliant occlusion balloon mounted at a proximal side thereof to the proximal catheter shaft and mounted at a distal side thereof to the distal catheter shaft;
a central catheter shaft extending through the proximal catheter shaft, the occlusion balloon and into the distal catheter shaft, the central catheter shaft constructed as the primary load-bearing chassis of the vascular occlusion catheter;
wherein the occlusion balloon defines a blown diameter between approximately twenty-five and approximately thirty-five millimeters and the occlusion balloon defines a double-wall thickness of between approximately 0.0003″ and approximately 0.0020″.

2. The vascular occlusion catheter of claim 1, wherein the occlusion balloon defines a functional working length of approximately 80 mm or less.

3. The vascular occlusion catheter of claim 1, wherein the proximal catheter shaft, the occlusion balloon, and the distal catheter shaft have a greatest outer diameter of seven French (7 Fr) or less in an uninflated condition.

4. The vascular occlusion catheter of claim 1, wherein upon inflation of the occlusion balloon into apposition with the target blood vessel, the occlusion balloon is configured to automatically adjust shape in response to diametric autoregulation of the target blood vessel, and, thereby, continue in apposition therewith.

5. The vascular occlusion catheter of claim 1, wherein upon inflation of the occlusion balloon into apposition with the target blood vessel, the occlusion balloon is configured to undergo intra-balloon inflation medium volume redistributrion, without altering intra-balloon inflation medium volume, in response to diametric autoregulation of the target blood vessel, and, thereby, automatically continue in apposition therewith.

6. The vascular occlusion catheter of claim 1, wherein upon inflation of the occlusion balloon into apposition with the target blood vessel, the occlusion balloon is configured to automatically transition from defining a generally cylindrically-shaped mid-portion bookended by generally conically-shaped portions to a tear-drop shape at a downsream end of the occlusion balloon in response diametric autoregulation of the target blood vessel into a normotensive state.

7. The vascular occlusion catheter of claim 1, wherein the occlusion balloon is configured such that an outer surface of the occlusion balloon comes into full diametric apposition with an inner surface of the target blood vessel upon partial inflation of the occlusion balloon, whereby folds are formed in the outer surface of the occlusion balloon, the folds defining flow channels with inner surfaces of the target blood vessel or with portions of the outer surface of the occlusion balloon that allow partial blood flow past the occlusion balloon.

8. A method of using the vascular occlusion catheter of claim 1, comprising the steps of:
inserting the vascular occlusion catheter in an uninflated condition into a desired location of the target blood vessel; and
inflating the occlusion balloon, with an appropriate volume of inflation medium, into a partially inflated state, wherein an outer surface of the occlusion balloon comes into substantially full diametric apposition with an inside wall of the target vessel and a state of partial occlusion of the target blood vessel is established,
whereby the occlusion balloon is configured to autonomously, substantially maintain the state of partial occlusion in response to autoregulation of the target blood vessel.

9. The method of claim 8, wherein the inserting step comprises advancing the distal catheter shaft, the occlusion balloon and the proximal catheter shaft through an introducer sheath having an inner introducer diameter of 7 Fr or less.

10. The method of claim 8, wherein the inflating step comprises inflating the occlusion balloon to an intra-balloon pressure of between approximately 60 mmHg and approximately 150 mmHg.

11. The method of claim 8, wherein the inflating step comprises folds being formed in the outer surface of the occlusion balloon, the folds defining flow channels with inner surfaces of the target blood vessel or with portions of the outer surface of the occlusion balloon that allow partial blood flow past the occlusion balloon.

12. The method of claim 8, wherein the occlusion balloon is configured to autonomously, substantially maintain the state of partial occlusion in response to autoregulation of the target blood vessel via automatically adjusting shape in response to diametric autoregulation of the target blood vessel.

13. The method of claim 8, wherein the occlusion balloon is configured to autonomously, substantially maintain the state of partial occlusion in response to autoregulation of the target blood vessel via intra-balloon inflation medium volume redistributrion, without altering intra-balloon inflation medium volume.

14. The method of claim 8, wherein the occlusion balloon is configured to autonomously, substantially maintain the state of partial occlusion in response to autoregulation of the target blood vessel via automatically transitioning from defining a generally cylindrically-shaped mid-portion bookended by generally conically-shaped portions to a tear-drop shape at a downsream end of the occlusion balloon.
